(19) United States
(12) Patent Application Publication
(10) Pub. No.: US 2021/0239610 A1
(43) Pub. Date: Aug. 5, 2021

(54) CONTAMINATION ANALYSER

(71) Applicant: eralytics GmbH, Wien (AT)

(72) Inventor: Niklas Christensson, Wien (AT)

(21) Appl. No.: 17/161,049

(22) Filed: Jan. 28, 2021

(30) Foreign Application Priority Data
Jan. 31, 2020 (AT) A 50080/2020

(51) Int. Cl. G01N 21/3577; G01N 1/40

(52) U.S. Cl. CPC G01N 21/3577; G01N 1/405

(57) ABSTRACT

A device for determining a concentration of a substance in a sample, comprising a sample reservoir, a measuring cell, a measuring channel, pressure application means, a computing unit as well as an infrared measurement setup, wherein the measuring channel is configured to remove the sample from the sample reservoir and to feed it to the measuring cell, wherein the device has a first solid phase extraction cartridge having a first sorbent and a second solid phase extraction cartridge having a second sorbent, wherein the two solid phase extraction cartridges are arranged between the sample reservoir and the measuring cell in such a way as to connect the sample reservoir respectively via a solid phase extraction cartridge to the measuring cell, wherein the first sorbent is configured to substantially completely adsorb/absorb the substance, the concentration of which is to be determined, and wherein the second sorbent is configured to substantially completely adsorb/absorb substances having a higher polarity (P) than the substance, the concentration of which is to be determined.

The present invention relates to a device for determining a concentration of a substance in a liquid sample, wherein the sample has a plurality of substances, comprising a sample reservoir, a measuring cell, a measuring channel, pressure application means, a computing unit as well as an infrared measurement setup, which has an infrared source as well as an infrared sensor for detecting measurement signals of an infrared beam having at least one predefined wavelength range, emitted by the infrared source and transmitting through the measuring cell, wherein the measuring channel is configured to remove the sample to be measured from the sample reservoir and feed it to the measuring cell by means of the pressure application means and wherein the computing unit is configured to analyse the measurement signals.

The invention further relates to a method for determining a concentration of a substance in a liquid sample.

Infrared spectroscopy or IR spectroscopy is a versatile technique for the rapid determination of the composition of liquids, based on the individual infrared characteristics of different molecules. However, the larger the molecules, the sample is composed of, the less specific the IR spectrum becomes, and it is largely determined by functional groups of the molecules, for example carbonyl groups in the case of esters. In this way, different compounds having similar functional groups, for example, having C═O bonds in esters and ketones, will absorb within the same IR spectral range, leading to overlapping of lines in the IR spectrum. Such overlapping of infrared spectral lines of similar or similarly functional groups, which are also designated as interferences, will lead to enormous measurement inaccuracies, due to which it is difficult or even impossible to measure very small concentrations of contaminations in a complex liquid, which is composed of many different molecules, such as in fuels, by means of IR spectroscopy.

This is in particular of a problem in the determination of the composition of aircraft fuels, which have a small proportion of biodiesel as contamination. Biodiesel, designated also as FAME (fatty acid methyl ester) as an umbrella term for methyl esters of different fatty acids, may only be present up to a critical value of 50 mg/kg in aircraft fuel, due to its disadvantageous properties on air traffic at low temperatures, which are given at the travel height of a passenger aircraft. Due to the required high measurement accuracy in the determination of the FAME content of aircraft fuels it is, hence, essential to avoid any measurement inaccuracies caused by overlapping of lines in the IR spectrum in the determination of the FAME content by way of IR spectroscopy.

In order to reduce such overlapping of IR spectral lines, there have been known several solution approaches, which, however, have either been too complex so far and/or cannot ensure a satisfying accuracy of the separation effect of the substances present in the liquid.

One way to reduce overlapping has been known, for example, from the patent application EP 3 454 045 A1, consisting in subjecting the sample to be measured to a chemical reaction using an additive, e.g. a solvent, wherein the sample is modified by the chemical reaction and in the course of the IR spectroscopy an altered concentration of the modified sample, corresponding to the addition of the additive, is determined. From the comparison of the concentration of the original and non-modified sample with the modified sample, hence, there may be drawn conclusions regarding the concentration of the substance to be measured in the sample.

The disadvantage of this method is that handling the measurement system is complex. Due to the use of harmful solvents in this method there is further to be paid attention to the health and safety aspects. Furthermore, the chemical reaction used in the method is not exclusively specific for FAME. Other molecules with carbonyl groups may also contribute to the measurement signal (for example, triglycerides). Furthermore, there is a risk of obtaining further interfering signals due to the added additives, which is why there is given an insufficient separation effect of the individual substances in the liquid. Furthermore, such a measurement takes a lot of measuring time with an average duration of twenty minutes.

Another way of reducing overlapping of IR spectral lines has been known from the patent document GB 2466802 A. The patent publication discloses a device for the determination of the concentration of a sub-substance of a liquid sample, wherein the sample to be examined is passed through a solid phase extraction cartridge before the concentration measurement. Solid phase extraction is a sample preparation method related to the possible enrichment, concentration or isolation of an analyte. It is a physical extraction process that takes place between a liquid sample, which is passed through the solid phase extraction cartridge, and a solid phase, which is also referred to as a sorbent, wherein the term solid phase extraction is broader and includes multiple sorbent/sample interactions, for example, polar and nonpolar interactions, ionic interactions, interactions via covalent bonds as well as multiple interactions.

In the device described in the patent document GB 2466802 A, a solid phase extraction cartridge, in a first step, filters substantially all polar sub-substances from the sample to be measured and passes the filtered sample further into a measuring cell. There, a reference spectrum is determined from the filtered sample. In a second step, a spectrum of the unfiltered sample is being determined. Then the spectrum of the filtered sample is compared to the spectrum of the unfiltered sample. From the comparison, conclusions may be drawn regarding the concentration of the substance to be determined in the sample.

The disadvantage of this method is that the measurement signal of the filtered sample is compared to the measurement signal of the unfiltered sample containing all substances. Therefore, there is no way of separating the substance to be determined from the other substances in the liquid sample using a device, which is why there is present an insufficient separation effect of the individual substances. This increases the susceptibility to false-positive results when evaluating the measurement signals. Such false-positive measurement results may make necessary, for example in the case of an apparently detected excess of the critical values of FAME in the determination of a FAME concentration in an aircraft fuel, subsequent examinations of the sample. These are time-consuming as well as cost-intensive, as they usually have to be carried out using highly precise and complex measurement methods, for example gas chromatography methods, due to the high demands on measurement accuracy.

Thus there is still a demand for an increased separation effect of substances to be prevented when determining the concentration of a substance in a liquid sample by way of IR spectroscopy.

The task of the invention is therefore to provide a device as well as a method, which avoid the disadvantages of the prior art.

These tasks are solved by providing a device for determining a concentration of a substance in a liquid sample having the features of claim 1 and by providing a method for determining a concentration of a substance in a liquid sample according to claim 5.

The device according to the invention comprises a first solid phase extraction cartridge having a first sorbent and a second solid phase extraction cartridge having a second sorbent, wherein the two solid phase extraction cartridges are connected to the measuring channel and are arranged between the sample reservoir and the measuring cell in such a way as to connect the sample reservoir to the measuring cell via a respective solid phase extraction cartridge, wherein the first sorbent is configured to substantially completely adsorb/absorb the substance, the concentration of which is to be determined, as well as substances having a higher polarity than the substance, the concentration of which is to be determined, and wherein the second sorbent is configured to substantially completely adsorb/absorb substances having a higher polarity than the substance, the concentration of which is to be determined.

This has the advantage that the substance to be determined may be isolated for further analysis thereof by passing unhindered through the second solid phase extraction cartridge but being retained by the first solid phase extraction cartridge. Thus, by comparing the infrared spectra obtained from the measured values of the sample, specific molecules within a very narrow range of properties, such as their polarity, can be isolated in the sample and subsequently analysed with high measurement accuracy. A measurement signal detected by the infrared measurement setup, after the liquid sample has passed through the first solid phase extraction cartridge, acts as a reference signal, which substantially contains only signal proportions of the apolar substances in the liquid sample. A measurement signal, on the other hand, which is detected after the liquid sample has passed through the second solid phase extraction cartridge, additionally also contains signal proportions of the polar substance to be determined, while substances having a higher polarity than the polarity of the substance to be determined are substantially completely adsorbed/absorbed by the second sorbent. Due to the thus purely apparatus-related increased separation effect, the concentration to be determined of the substance may be established with a much higher accuracy than with previously known measuring devices, which is why the device according to the invention is suited for compliance with the required product specifications of aircraft fuels in refineries, terminals and airports.

According to a preferred embodiment the material, the first sorbent is composed of, is selected from a magnesium silicate or a combination of magnesium silicates, in particular Florisil.

By using Florisil as a sorbent, polar molecules may advantageously be separated from apolar molecules, wherein in the case of Florisil substantially polar interactions are responsible for adsorption/absorption. When a liquid containing polar and apolar molecules is passed through the first solid phase extraction cartridge with Florisil as sorbent, the polar molecules are adsorbed, while the apolar molecules pass unaffected. In this way, the measurement signal obtained after IR analysis may be used as a reference signal for determining the concentration of the substance to be determined.

According to a further preferred embodiment the material, the second sorbent is composed of, is selected from an alkyl amine, in particular from an amino propyl.

Because of the selection of this sorbent material, molecules of the liquid having lower polarity than a target polarity determined by the selected material may pass undisturbed through the solid phase extraction cartridge when passing through the second solid phase cartridge. Molecules having higher polarity than the target polarity are adsorbed by the cartridge. In particular molecules of the functional group of esters may pass unhindered, while molecules having higher polarity than the target polarity, for example ketones and acids, are adsorbed/absorbed.

According to a further preferred embodiment the pressure application means are pumps, wherein respectively one pump is connected to one of the two solid phase extraction cartridges.

This has the advantage that the liquid sample may be easily sucked from the sample reservoir by means of suction by a first pump through the first solid phase extraction cartridge and by means suction by a second pump through the second solid phase extraction cartridge and subsequently fed to the infrared measurement setup. In this way, there is no need for the use of an additionally to be operated switching element, for example a valve, in the measuring channel of the device, which would have to be additionally controlled or manually operated.

According to another preferred embodiment of the invention the liquid sample is an aircraft fuel and the substance, the concentration of which is to be determined, is FAME or biodiesel. In this way, the biodiesel concentration in an aircraft fuel may be determined by means of the device according to the invention.

The method according to the invention comprises the following steps: Feeding the liquid sample from the sample reservoir via the first solid phase extraction cartridge into the measuring cell by means of the pressure application means;

Detecting a first measurement signal by detecting an infrared beam, transmitting through the measuring cell and emitted by the infrared source, of a first wavelength range by the infrared sensor;

Feeding the liquid sample from the sample reservoir via the second solid phase extraction cartridge into the measuring cell by means of the pressure application means;

Detecting a second measurement signal by detecting an infrared beam, transmitting through the measuring cell and emitted by the infrared source, of the first wavelength range by the infrared sensor;

Determining a first difference signal from the difference of the second measurement signal and the first measurement signal by the computing unit;

Determining a first concentration of the substance in the liquid sample from the first difference signal by the computing unit.

Thus, there is obtained the advantage that the concentration of the substance to be determined from the liquid sample may be determined from the difference of the two measurement signals, wherein signal interferences, which occur due to different compounds having similar functional groups in the liquid sample, are eliminated by the difference formed by the two measurement signals. In particular, the measuring device according to the invention for determining the concentration of a substance in a liquid sample, which carries out this method, may determine the concentration to be determined much faster than previously known measuring devices. In particular, compared to methods, in which the liquid sample is modified by means of a chemical reaction, a significant reduction in the measurement time may be achieved.

According to a preferred embodiment of the method, the method comprises the following further steps:

Feeding the liquid sample from the sample reservoir via the first solid phase extraction cartridge into the measuring cell by means of the pressure application means;

Detecting a third measurement signal by detecting an infrared beam, transmitting through the measuring cell and emitted by the infrared source, of a second wave length range by the infrared sensor;

Feeding the liquid sample from the sample reservoir via the second solid phase extraction cartridge into the measuring cell by means of the pressure application means;

Detecting a fourth measurement signal by detecting an infrared beam, transmitting through the measuring cell and emitted by the infrared source, of the second wave length range by the infrared sensor;

Determining a second difference signal from the difference of the fourth measurement signal and the third measurement signal by the computing unit;

Determining a second concentration of the substance in the liquid sample from the second difference signal by the computing unit in order to establish a predefined level of accordance between the first concentration of the substance and the second concentration of the substance.

Thus, by comparing the second concentration of the substance from the second difference signal with the first concentration of the substance from the first difference signal, it is possible to examine in a simple manner whether the first concentration determined in the first wavelength range matches the second concentration determined in the second wavelength range within the frame of the predefined level of accordance and whether substantially all interferences have been eliminated by the solid phase extraction. The separation effect of the detection of the substance is thus further increased and the validity of the measurement result may thus be examined particularly efficiently. If the two concentrations determined in this way lie within the limits of measurement accuracy, the actual concentration of the analyte will thus be obtained. The established concentration of the substance to be determined may be presented to the user in the following, for example, via a display.

In particular, by determining the predefined level of accordance between the first concentration of the substance and the second concentration of the substance by the computing unit, it is possible to measure the concentration of FAME in aircraft fuels with a detection limit of 10 mg/l, without the result being falsified by interference with oxidation products and/or additives. It has not been possible so far to detect such interferences using previously known methods.

According to a further preferred embodiment of the method, the detection of the first and third measurement signals is carried out simultaneously, and the detection of the second and fourth measurement signals is carried out simultaneously.

In this way, the verification of the validity of the measurement result may advantageously be carried out in a particularly time- and cost-saving manner, whereby the simultaneous detection of the measurement signals may be realised in a known manner by a suitable infrared measurement setup, for example by using infrared sensors, which have a plurality of sensor surfaces.

According to a further preferred embodiment of the method, the method comprises the following further steps:

Determining a correction difference signal from the first difference signal and the second difference signal upon a first deviation from the predefined level of accordance between the first concentration of the substance and the second concentration of the substance by the computing unit;

Determining a corrected concentration of the substance in the liquid sample from the correction difference signal by the computing unit.

This has the advantage that, in the event of only a slight deviation of the first concentration from the second concentration, a corrected concentration of the substance in the liquid sample may be established particularly easily by applying a correction term, which may be taken from a table of correction terms, for example, without impairing the required measurement accuracy. The corrected concentration of the substance to be determined may subsequently be presented to the user, for example via a display.

According to a further preferred embodiment of the method, the method comprises the following further steps:

Identifying a further substance upon a second deviation from the predefined level of accordance between the first concentration of the substance and the second concentration of the substance.

Thus, in case of a too high deviation of the first concentration from the second concentration, at which a correction of the concentration is no longer possible, it may advantageously be recognized by the device that the examined liquid sample is not the sought-after substance but rather that an unknown substance is present. The established concentration of the unknown substance may then be presented to the user on a display, for example by way of a message indicating the presence of the unknown substance.

The present invention will now be explained in greater detail by means of examples of embodiments with reference to the figures.

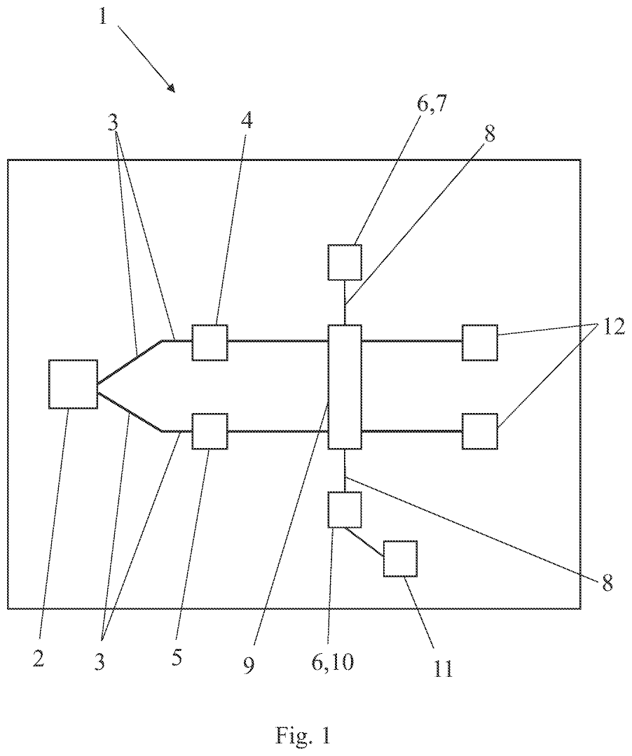

FIG. 1 shows a schematic depiction of the essential components of a device for determining a concentration of a substance in a liquid sample according to claim 1.

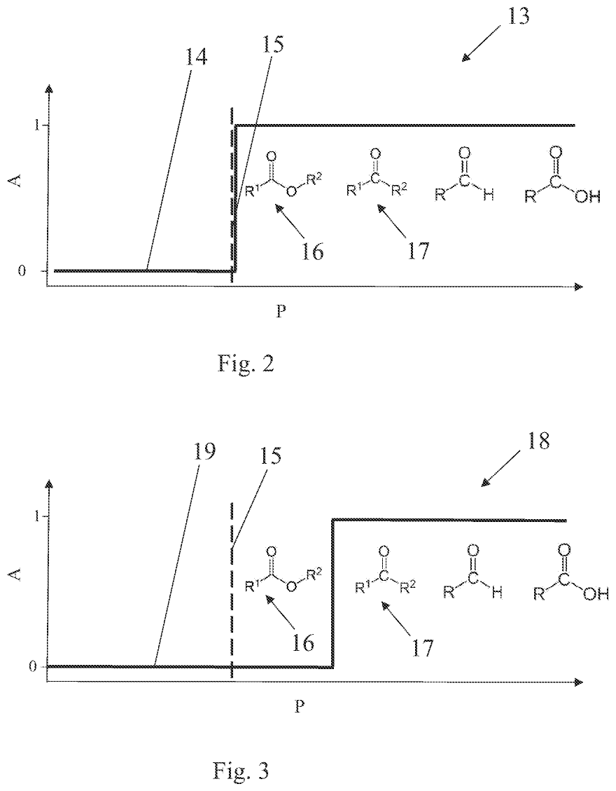

FIG. 2 shows a diagram, which illustrates the course of the absorption coefficient of the first sorbent of the first solid phase extraction cartridge as a function of the polarity of the liquid to be measured.

FIG. 3 shows a diagram, which illustrates the course of the absorption coefficient of the second sorbent of the second solid phase extraction cartridge as a function of the polarity of the liquid to be measured.

FIG. 1 shows a device 1 for determining a concentration of a substance in a liquid sample, which contains a plurality of substances. The device 1 comprises a sample reservoir 2, from which, via a measuring channel 3, a liquid sample is removed by pressure application means 12 and fed to a measuring cell 9.

The pressure application means 12 may be configured as suction means or pressure means. According to a preferred embodiment of the device 1, the pressure application means 12 in FIG. 1 are configured as pumps, wherein respectively one pump is connected to one of the two solid phase extraction cartridges 4, 5. Thus one of the two pumps is connected to the first solid phase extraction cartridge 4 via the measuring channel 3. The other of the two pumps is connected to the second solid phase extraction cartridge 5 via the measuring channel 3. Depending on which of the pumps is used to remove the liquid sample from the sample reservoir 2 via the measuring channel 3, the liquid sample is routed via one of the two solid phase extraction cartridges 4, 5.

Alternatively or in addition thereto, the device 1 may also have several pumps and solid phase extraction cartridges 4, 5, wherein this embodiment is not shown in FIG. 1.

When the liquid sample passes the first solid phase extraction cartridge 4 or the second solid phase extraction cartridge 5, part-substances of the liquid sample will be removed from the liquid sample by adsorption or absorption according to the properties of the respective sorbent and remain in one of the two respective solid phase extraction cartridges 4, 5.

After the liquid sample has passed through the first solid phase extraction cartridge 4 or the second solid phase extraction cartridge 5, it enters a measuring cell 9 in which, by means of an infrared measurement setup 6, which comprises an infrared source 7 as well as an infrared sensor 10, the infrared sensor 10 detects measurement signals of an infrared ray 8 of at least one predefined wavelength range, emitted by the infrared source 8 and illuminating and transmitting through the measuring cell 9.

The at least one predefined wavelength range is between 1 μm and 20 μm.

The measurement signal is finally evaluated by a computing unit 11.

The FIGS. 2 and 3 illustrate the process of separating specific molecules, in this case esters, based on their polarity by the two solid phase cartridges 4, 5. By selecting a suitable sorbent of the two solid phase extraction cartridges 4, 5, it is possible to selectively filter substances from the liquid sample on the basis of their chemical properties, for example their polarity, before the liquid sample enters the measuring cell 9 and the measurement signal thereof is detected. In this way, each of the two solid phase extraction cartridges 4, 5 has a respective target polarity, at which a sudden increase in adsorption/absorption by the respective sorbent takes place.

Thus, according to a preferred embodiment of the device 1, the material, the first sorbent of the first solid phase extraction cartridge 4 is composed of, is selected from a magnesium silicate or a combination of magnesium silicates, in particular Florisil. By choosing Florisil as the sorbent material of the first solid phase extraction cartridge 4, substantially all polar molecules contained in the liquid sample will be bound in the first solid phase extraction cartridge 4, while apolar molecules will pass undisturbed through the first solid phase extraction cartridge 4. Alternatively and/or additionally, any other material may be used as a sorbent, which results in substantially all polar molecules contained in the liquid sample being bound in the first solid phase extraction cartridge 4.

FIG. 2 shows in a diagram 13 a first absorption curve 14 of the absorption A of the first solid phase extraction cartridge 4, which contains Florisil as sorbent, as a function of the polarity P of the molecules of the liquid sample, which flow through the first solid phase extraction cartridge 4. The polarity-dependent absorption A is normalised in such a way that a value of one corresponds to a complete absorption of the substance by the sorbent of the solid phase extraction cartridge. As can be seen in FIG. 2, no molecule will be adsorbed/absorbed below a threshold polarity 15, which marks the transition between a polar and an apolar molecule and is shown as a dashed line in FIG. 2. Polar molecules, on the other hand, which have a polarity P that is higher than the threshold polarity 15, will be substantially completely bound by the sorbent in the first solid phase extraction cartridge 4, wherein the first absorption curve 14 rises abruptly at the target polarity of the first solid phase extraction cartridge 4 in the region of the threshold polarity 15. Such molecules are in particular molecules of the functional group of esters 16 as well as higher polar molecules 17, for example ketones, aldehydes and acids.

If, however, according to a further preferred embodiment of the device 1, the material, the second sorbent is composed of, is selected from an alkyl amine, in particular from an amino propyl, molecules having a lower polarity P than a predetermined target polarity pass undisturbed through the second solid phase extraction cartridge 5, while molecules having a higher polarity P than a selected target polarity are substantially completely adsorbed/absorbed by the second sorbent of the second solid phase extraction cartridge 5. Alternatively and/or complementarily, any other material may be used as a sorbent, which results in molecules having a higher polarity P than the target polarity being substantially completely adsorbed/absorbed by the second sorbent of the second solid phase extraction cartridge 5.

FIG. 3 shows in a diagram 18 a second absorption curve 19 of the absorption A of the second solid phase extraction cartridge 5, which has an alkyl amine, optionally with a chain length of 2 to 10 carbon atoms, in particular a propyl amine or butyl amine, as sorbent, as a function of the polarity P of the molecules of the liquid sample, which flow through the second solid phase extraction cartridge 5. The sudden rise in the absorption curve 19 now occurs at the target polarity of the second solid phase extraction cartridge 5 between the esters 16 and the higher polar molecules 17 such that esters 16 and molecules having a lower polarity P than esters will pass unhindered through the solid phase extraction cartridge, while ketones, aldehydes and acids will be bound in the second solid phase extraction cartridge 5 due to their higher polarity P in comparison to esters 16.

Due to the different absorption behaviour of the first absorption curve 14 of the first solid phase extraction cartridge 4 in FIG. 2 and the second absorption curve 19 of the second solid phase extraction cartridge 5 in FIG. 3, there is obtained an increased separation effect of the substance to be examined in purely technical terms in comparison to prior art.

When the sorbent of one of the solid phase extraction cartridges 4, 5 is saturated, the adsorbed molecules may be washed out of the solid phase extraction cartridges 4, 5 substantially completely by rinsing the solid phase extraction cartridges 4, 5 using polar solvents, as the absorption/adsorption process is reversible.

What is claimed is:

1. A device for determining a concentration of a substance in a liquid sample, wherein the sample has a plurality of substances, comprising a sample reservoir, a measuring cell, a measuring channel, pressure application means, a computing unit as well as an infrared measurement setup, which has an infrared source as well as an infrared sensor for detecting measurement signals of an infrared beam having at least one predefined wavelength range, emitted by the infrared source and transmitting through the measuring cell, wherein the measuring channel is configured to remove by means of the pressure application means the sample to be measured from the sample reservoir and to feed it to the measuring cell and wherein the computing unit is configured to analyse the measurement signals, characterized in that the device further has a first solid phase extraction cartridge having a first sorbent and a second solid phase extraction cartridge having a second sorbent, wherein the two solid phase extraction cartridges are connected to the measuring channel and are arranged between the sample reservoir and the measuring cell in such a way as to connect the sample reservoir respectively via a solid phase extraction cartridge to the measuring cell, wherein the first sorbent is configured to substantially completely adsorb/absorb the substance, the concentration of which is to be determined, as well as substances having a higher polarity (P) than the substance, the concentration of which is to be determined, and wherein the second sorbent is configured to substantially completely adsorb/absorb substances having a higher polarity (P) than the substance, the concentration of which is to be determined.

2. A device according to claim 1, characterized in that the material, the first sorbent is composed of, is selected from a magnesium silicate or a combination of magnesium silicates.

3. A device according to claim 1, characterized in that the material, the first sorbent is composed of, is selected from an alkyl amine, in particular from a propyl amine or butyl amine.

4. A device according to claim 1, characterized in that the pressure application means are pumps, wherein respectively one pump is connected to one of the two solid phase extraction cartridges.

5. A device according to claim 1, characterized in that the device is configured to process, as a liquid sample, an aircraft fuel and, as a substance, the concentration of which is to be determined, FAME or biodiesel.

6. A method for determining a concentration of a substance in a liquid sample by means of the device according to claim 1, wherein the method comprises the following steps:
Feeding the liquid sample from the sample reservoir via the first solid phase extraction cartridge into the measuring cell by means of the pressure application means;
Detecting a first measurement signal by detecting an infrared beam, transmitted through the measuring cell and emitted by the infrared source, of a first wavelength range by the infrared sensor;
Feeding the liquid sample from the sample reservoir via the second solid phase extraction cartridge into the measuring cell by means of the pressure application means;
Detecting a second measurement signal by detecting an infrared beam, transmitted through the measuring cell and emitted by the infrared source, of the first wavelength range by the infrared sensor;
Determining a first difference signal from the difference of the second measurement signal and the first measurement signal by the computing unit;
Determining a first concentration of the substance in the liquid sample from the first difference signal by the computing unit.

7. A method according to claim 6, wherein the method comprises the following further steps:
Feeding the liquid sample from the sample reservoir via the first solid phase extraction cartridge into the measuring cell by means of the pressure application means;
Detecting a third measurement signal by detecting an infrared beam, transmitted through the measuring cell and emitted by the infrared source, of a second wavelength range by the infrared sensor;
Feeding the liquid sample from the sample reservoir via the second solid phase extraction cartridge into the measuring cell by means of the pressure application means;
Detecting a fourth measurement signal by detecting an infrared beam, transmitted through the measuring cell and emitted by the infrared source, of the second wavelength range by the infrared sensor;
Determining a second difference signal from the difference of the fourth measurement signal and the third measurement signal by the computing unit;
Determining a second concentration of the substance in the liquid sample from the second difference signal by the computing unit in order to establish a predefined level of accordance between the first concentration of the substance and the second concentration of the substance.

8. A method according to the claim 6, wherein the detection of the first and the third measurement signal is carried out simultaneously and the detection of the second and fourth measurement signal is carried out simultaneously.

9. A method according to claim 7, wherein the method comprises the following further steps:
Determining a correction difference signal from the first difference signal and the second difference signal upon a first deviation from the predefined level of accordance between the first concentration of the substance and the second concentration of the substance by the computing unit;
Determining a corrected concentration of the substance in the liquid sample from the correction difference signal by the computing unit.

10. A method according to claim 7, wherein the method comprises the following further steps:
Identifying a further substance upon a second deviation from the predefined level of accordance between the first concentration of the substance and the second concentration of the substance.
